(19) United States
(12) Patent Application Publication
(10) Pub. No.: US 2007/0293866 A1
(43) Pub. Date: Dec. 20, 2007

(54) BONE ANCHOR SYSTEM AND METHOD OF USE

(76) Inventors: Dieter Stoeckel, Menlo Park, CA (US); Minh Dinh, Union City, CA (US); Tracey Lopes, Mason, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/747,807

(22) Filed: May 11, 2007

Related U.S. Application Data
(60) Provisional application No. 60/747,172, filed on May 12, 2006.

(51) Int. Cl. A61B 17/58

(52) U.S. Cl. 606/72

(57) ABSTRACT

The present invention relates to bone anchors, particularly of the type for fixing medical devices to bone. The bone anchor system includes a bone-anchoring element that has super elastic and/or shape memory components that extend radially outward for engaging the bone.

### CROSS-REFERENCE TO RELATED APPLICATIONS

This application claims the benefit of U.S. Provisional Application Ser. No. 60/747,172 filed May 12, 2006.

### FIELD OF THE INVENTION

The present invention relates to bone fixation systems and, more particularly, to bone anchors of the type for fixing medical devices to bone. Various embodiments of the present device may also be used to fix soft tissue or tendons to bone, or for securing two or more adjacent bone fragments or bones together.

### BACKGROUND OF THE INVENTION

In the art of orthopedic surgery, and particularly in spinal surgery, it has long been known to affix an elongated member, such as a plate or rod, to bones in order to hold them and support them in a given position. For example, in a procedure to fuse damaged vertebrae, the vertebrae are positioned in a corrected position as required by the surgeon. A plate is placed adjacent to the bone, and bone anchors are employed to secure the plate to the bones. Bone screws or bolts are commonly utilized as the bone anchors. With such anchors, placement is accomplished by drilling one or more holes in the bone(s), and threading the anchors into the holes. An example of a prior art bone bolt is described in a book by Dr. Cotrel entitled New Instrumentation for Surgery of the Spine. Freund, London 1986. An anchor can be threaded into a hole through the plate, or the plate can be placed in position around the anchor after threading into the hole. The anchor and plate are then secured to each other to prevent relative movement. In this way, bones may be held and/or supported in proper alignment for healing.

A spinal plate system or other similar implant system may have anchors that can be positioned at a number of angles with respect to the plate or other implant. Such a feature allows easier placement of implant systems or correction of positioning of an implant system, in that the bone anchors need not be precisely positioned in angular relation with respect to the implant. Rather, with a multi-axial capability, holes can be drilled in a bone at a convenient location and/or angle, for example, and screws can be inserted therein, with the connection between the plate and the anchor being angularly adjustable to provide sufficient force perpendicular to the plate/bone interface to secure the plate.

The plate system disclosed in U.S. Pat. No. 5,613,967 to Engelhardt, et al., discloses a slotted plate through which a bone screw extends. The screw includes cancellous threads for placement in bone, an intermediate section with an upper flat portion, and a machine-threaded section. The machine-threaded portion fits through the slot in the plate, and the plate abuts the flat portion of the screw or a flat washer imposed between the intermediate portion of the screw and the plate. A bracket is placed over the machine-threaded portion of the screw and the slotted plate, and a nut is threaded on the machine-threaded portion of the screw to anchor the screw and plate together. This apparatus does not provide the preferred multi-axial capability, as described above.

U.S. Pat. No. 5,084,048 to Jacob et al., discloses apparatus for clamping a rod to a bone screw such that the longitudinal planes of the rod and screw are not perpendicular.

Bones that have been fractured, either by accident or severed by surgical procedure, must be kept together for lengthy periods of time in order to permit the recalcification and bonding of the severed parts. Accordingly, adjoining parts of a severed or fractured bone are typically clamped together or attached to one another by means of a pin or a screw driven through the rejoined parts. Movement of the pertinent part of the body may then be kept at a minimum, such as by application of a cast, brace, splint, or other conventional technique, in order to promote healing and avoid mechanical stresses that may cause the bone parts to separate during bodily activity.

Bone anchors can also be used to attach fibrous tissues, such as ligaments and tendons that have detached from bones. For example, it is known to fix a fibrous tissue to bone by inserting a suture anchor through the fibrous tissue and into the bone, and then knotting the suture attached to the anchor in order to tie down the fibrous tissue to the bone. One embodiment of the present invention may be used to anchor such suture anchor to the bone.

Notwithstanding the variety of bone fasteners that have been developed in the prior art, there remains a need for a bone fastener of the type that can accomplish shear-force stabilization with minimal trauma to the surrounding tissue both during installation and following bone healing.

In addition, there remains a need for a simple, bone fixation device that may be utilized to secure medical devices or bone to bone.

### BRIEF DESCRIPTION OF THE INVENTION

The present invention relates to fixation systems and, more particularly, to anchors of the type for fixing medical devices to bone.

In one embodiment, the present invention includes a bone anchor assembly comprising an anchor core having a proximal and distal end, and an elongate tubular anchor element concentrically disposed over and engaged with the anchor core. The anchor element includes shape set protrusions extending radially outward for engaging with a bone.

In another embodiment, the present invention includes an anchor assembly comprising an anchor core, and an anchor element concentrically disposed over and engaged with the anchor core. The anchor element includes shape set protrusions extending radially outward for engaging with a recess.

In a further embodiment, the present invention includes a method of fixating a bone anchor assembly comprising the steps of making a hole sized to operably accept the anchor assembly in bone, the anchor assembly including a plurality of shape set protrusions; inserting the anchor assembly into the opening of the hole without tapping threads into the wall of said hole; linearly inserting the anchor assembly until the shape set protrusions are operably engaged with the inner surface of the hole; and securing a plurality of medical devices to the distal portion of the anchor assembly.

In yet a further embodiment, the present invention includes a method of using the anchor assembly, the anchor assembly having at least one shape set protrusion, comprising the steps of making a hole in a solid material sized to operably accept the anchor assembly; linearly inserting the anchor assembly into the opening of the hole without tapping threads into the wall of the hole until the at least one shape set protrusion is operably engaged with the inner surface of the hole.

### BRIEF DESCRIPTION OF THE DRAWINGS

FIG. 1 is a perspective view of an anchor assembly according to one embodiment of the present invention.

FIG. 2 is a perspective exploded view of components comprising the anchor assembly according to one embodiment of the present invention.

FIG. 3 is a perspective view of a laser cut tube prior forming the anchor by shape setting according to one embodiment of the present invention.

FIG. 4A is a side view of the anchor according to one embodiment of the present invention.

FIG. 4B is a perspective view of the anchor according to one embodiment of the present invention.

FIG. 5 is a perspective view of the anchor assembly, including an axial head, according to one embodiment of the present invention.

### DETAILED DESCRIPTION

The present invention relates to bone fixation systems and, more particularly, to bone anchors of the type for fixing medical devices to bone. Although a bone anchor used for repair of the spine is described for the purpose of example, one of skill in the art would understand that other embodiments of this device could be used to fix soft tissue or tendons to bone, or for securing two or more adjacent bone fragments or bones together. Still, one of skill in the art would understand that embodiments of the present invention may be used to fix other materials, or to fix other devices to a variety of materials.

Spinal fracture fixation is surgically accomplished through internal fixation utilizing metal implants. Bone screws are one part of spinal fixation systems that allows mobility of the patient while treating damaged bone. The screws may be used to reclaim functionality lost due to osteoporotic fractures, traumatic injuries, or disc herniations. The success of a bone screw is measured by its ability to not only purchase the fractured bone but also to adhere and integrate into the bone structure, providing a secure, long-term implant.

The basic principles of prior art bone screws are for the threads to match with a solid material to provide a strong interface. When the material is porous, such as in the case of osteoporosis (>95% porosity), pullout resistance is significantly decreased.

Previous modifications made to existing bone screw designs often failed to yield statistical increases in pullout strength. Doubling the threads of a screw showed no significant increase in pullout resistance. Some bone anchor systems that tried to overcome inadequate pullout strength incorporated a hollow modular anchorage system that allowed the delivery of cement through the end of the screw. This system also failed to improve the pullout strength. In an attempt to increase the osteointegration of screws, biomaterials have been used in the fabrication of the implants. While improved osteointegration was demonstrated, pullout strength has been reported to decrease by as much as 60%. Although implant material properties closer to the native bone as well as architecture more closely designed to the tissue may aid in osteointegration, current bone screw designs have not shown long-term success of bone fractures requiring fixation.

Existing bone anchor systems generally work by screwing a bone anchor into a predrilled, and sometimes pre-tapped hole. Manual bone anchor placement devices include a lever, a force translator and a rotary force mechanism. The devices are substantially gun or pistol-shaped and are actuated when a user squeezes the lever to the gripping portion of a handle. Manual, linear force on the lever is mechanically translated through the force translator to the rotary force mechanism, which in turn transmits a rotary force to a securing element, or coupler. The securing element mates with a bone anchor screw. The rotation of the securing element or coupler applies a torque on the bone anchor screw thereby placing the screw into bone.

To overcome these and other problems, the present invention allows the anchoring element to easily collapse into a low profile that creates a minimum insertion force when the anchor is inserted into a core hole drilled into a bone. This unique design does not require the core hole to be pre-tapped, which virtually eliminates torque application to the bone prior to and during anchor insertion.

In a preferred embodiment, the present invention includes bone-anchoring elements that have super elastic and/or shape memory qualities for enhanced performance. One example of a shape memory metal is Nickel Titanium (Nitinol).

Nitinol is utilized in a wide variety of applications, including medical device applications as described above.

Nitinol or NiTi alloys are widely utilized in the fabrication or construction of medical devices for a number of reasons, including its biomechanical compatibility, its bio-compatibility, its fatigue resistance, its kink resistance, its uniform plastic deformation, its magnetic resonance imaging compatibility, its ability to exert constant and gentle outward pressure, its dynamic interference, its thermal deployment capability, its elastic deployment capability, its hysteresis characteristics, and its moderate radiopacity.

Nitinol, as described above, exhibits shape memory and/or super elastic characteristics. Shape memory characteristics may be simplistically described as follows.

A metallic structure, for example, a Nitinol tube that is in an Austenitic phase may be cooled to a temperature such that it is in the Martensitic phase. Once in the Martensitic phase, the Nitinol tube may be deformed into a particular configuration or shape by the application of stress. As long as the Nitinol tube is maintained in the Martensitic phase, the Nitinol tube will remain in its deformed shape. If the Nitinol tube is heated to a temperature sufficient to cause the Nitinol tube to reach the Austenitic phase, the Nitinol tube will return to its original or programmed shape. The original shape is programmed to be a particular shape by well-known techniques.

Super elastic characteristics may be simplistically described as follows. A metallic structure for example, a Nitinol tube that is in an Austenitic phase may be deformed to a particular shape or configuration by the application of mechanical energy. The application of mechanical energy causes a stress induced Martensitic phase transformation. In other words, the mechanical energy causes the Nitinol tube to transform from the Austenitic phase to the Martensitic phase. Once the mechanical energy or stress is released, the Nitinol tube undergoes another mechanical phase transformation back to the Austenitic phase and thus its original or programmed shape. By utilizing the appropriate measuring instruments, one can determine that the application or release of mechanical energy (stress) causes a temperature increase or temperature drop, respectively, in the Nitinol tube. As described above, the original shape is programmed by well know techniques. The Martensitic and Austenitic phases are common phases in many metals.

Medical devices constructed from Nitinol are typically utilized in both the Martensitic phase and/or the Austenitic phase. The Martensitic phase is the low temperature phase. A material that is in the Martensitic phase is typically very soft and malleable. These properties make it easier to shape or configure the Nitinol into complicated or complex structures. The Austenitic phase is the high temperature phase. Nitinol in the Austenitic phase is generally much stronger than the Nitinol in the Martensitic phase. Typically, many medical devices are cooled to the Martensitic phase for manipulation and loading into delivery systems. When the device is deployed at body temperature, the concomitant change in temperature drives the device toward a return to the Austenitic phase.

Although Nitinol is described in this embodiment, it should not be understood to limit the scope of the invention. One of skill in the art would understand that other materials, both metallic and pseudo-metallic exhibiting similar shape memory and super-elastic characteristics may be used.

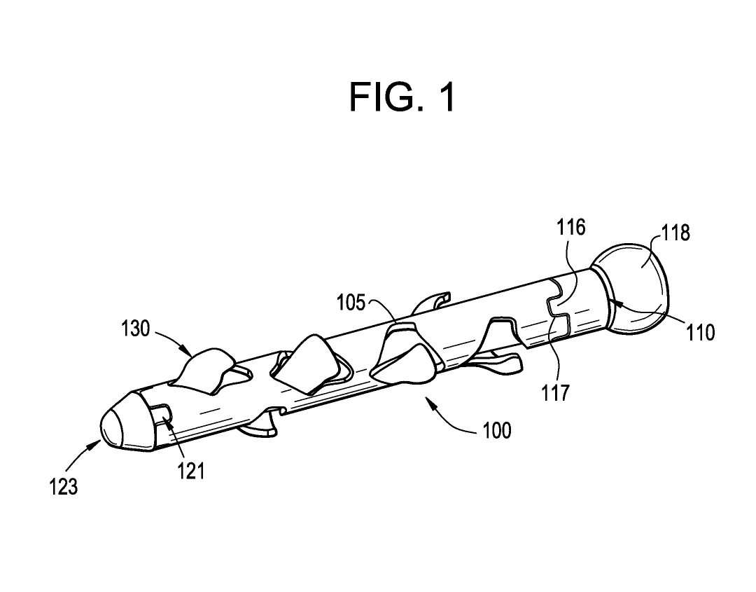

The anchoring system 100 of the present invention includes two basic components, an anchoring element and an anchor core. FIG. 1 is a perspective view of an anchor assembly 100 illustrating the anchor element 105 and the anchor core 110 according to one embodiment of the present invention.

The anchor element 105 is made from a metallic or pseudo-metallic tube having super-elastic properties. In a preferred embodiment, the anchor element 105 is made from a nickel titanium alloy, such as Nitinol.

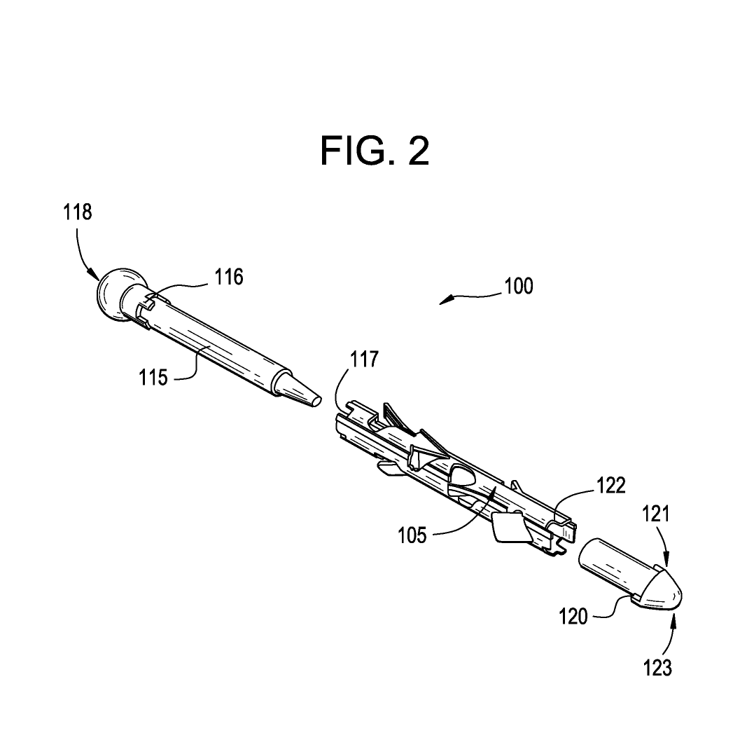

The anchor core 110 is sized to engage and support the anchor element 105, where such support may optionally be radial, axial, or both radial and axial. Further, the anchor core 110 may be sized to secure the anchor element to a coupler or axial head. In one embodiment of the invention, the anchor core 110 is comprised of a proximal core 115 and a distal core 120. FIG. 2 is an exploded perspective view illustrating the relationship between the anchor element 105 and anchor core 110 components 115, 120 according to one embodiment of the present invention. As can be seen, the proximal and distal anchor cores 115, 120 respectively have stepped profiles. With the exception of the extreme proximal end 118 of the proximal core 115 and the extreme distal end 123 of the distal core 120, the outside diameters are generally smaller than the inside diameter of the anchor element 105. This allows the anchor cores 115, 120 to pass through the inside of the anchor element 105 to support and add rigidity to the anchor element 105. In addition, the distal end of the proximal core 115 and proximal end of the distal core 120 may also have mating opposing ends to facilitate the convergence of these components. This configuration will further add to the rigidity of the anchor core 110 and support of the anchor element 105.

In the illustrated embodiment, the distal core 120 has a conically shaped distal tip 123 to assist in locating and deploying the distal end of the anchor system 100 in a core hole in the target bone. The distal core 120 may additionally incorporate a cog 121 sized to engage a detent 122 formed into the distal end of the anchor element 105.

The proximal end of the proximal core 115 may be shaped to facilitate attachment of anchor assembly 100 to a deployment device or medical device such a polyaxial head, as is known in the art. In one embodiment of the invention, the proximal end of the proximal core 115 has a spherical shape to accept an axial head.

As described above, the proximal core 115 may incorporate a cog 116 sized to engage a detent 117 formed into the proximal end of the anchor element 105. These cogs and detents fix the proximal and distal anchor core element 115, 120 to the anchor element 105, allowing any rotational energy applied to the core elements 115, 120 to be transmitted to the anchor element 105.

The anchor core 110 elements 115, 121 may be made of any biocompatible material with sufficient strength, such as, for example, stainless steel or Titanium.

The anchor element 105 has a series of special leaves 130 that are cut from the Nitinol tube, and then shape set to a normal open configuration. That is to say, the shape of the leaves are cut in the tube, and then the leaves are bent out and shape set in the desired configuration, taking full advantage of the super elastic and/or shape memory characteristics of the material.

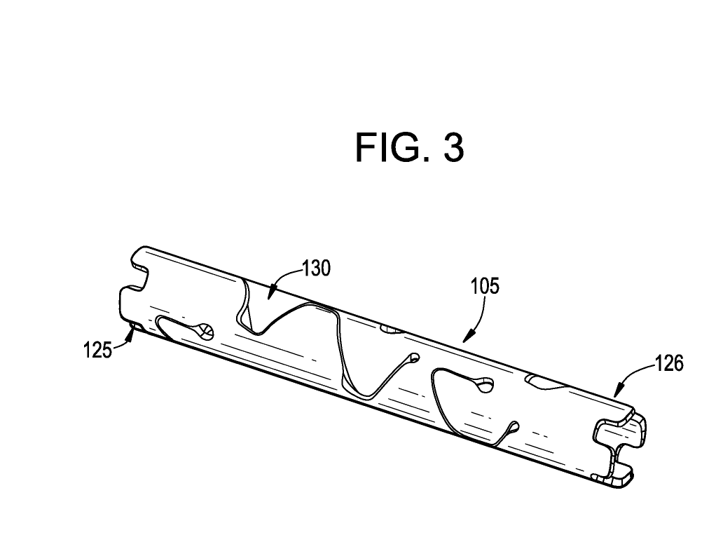

FIG. 3 is a perspective view of a Nitinol tube used to make the anchor element 105 according to one embodiment of the present invention. The leaves 130 may be cut in the Nitinol tube by any method known to one skilled in the art, such as by mechanical, water jet, or chemical means. In a preferred embodiment, the leaves 130 are cut in the Nitinol tube by a laser. As can be seen, the leaves 130 are cut on three sides to the desired pattern. Once the leaves 130 are completely cut in the tube, they are bent open to the desired configuration and shape set to resiliently retain their position.

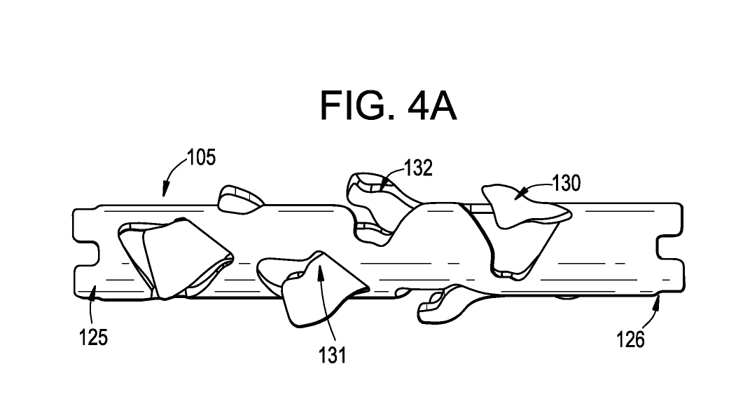

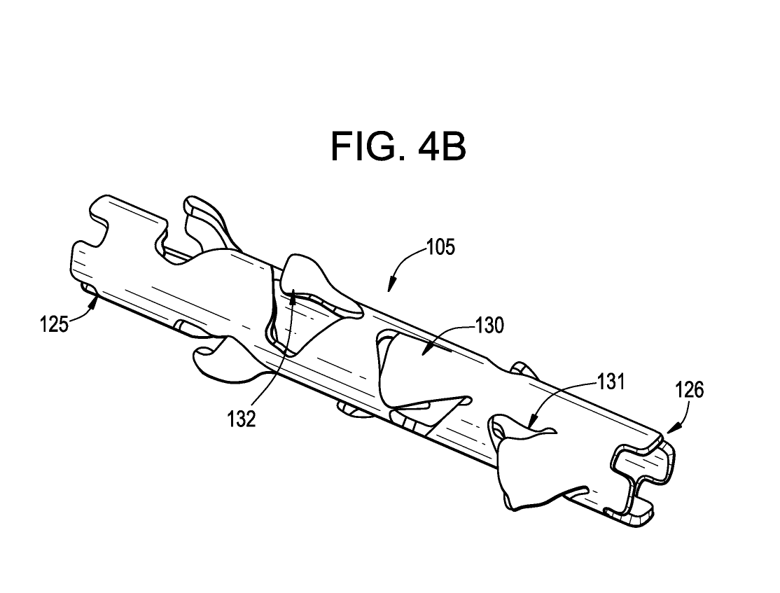

FIGS. 4A and 4B are side and perspective views respectively of anchoring element 105 according to one embodiment of the present invention. As can be seen, the anchoring element 105 includes a series of leaves 130 laser cut from the super elastic Nitinol tube in a spiral configuration. The super elastic leaves 130 are shape set in the normal open position so that all leaves are extended out from the tube's outer circumference. The super elastic properties of the anchor element 105 allows the leaves 130 to be compressed back into the closed, pre set position when the anchor assembly 100 is inserted into the bone.

The leaves 130 are shown cut from the tube in a spiral configuration. That is to say, adjacent leaves 130 are rotationally offset from one another as they progress from the distal end 126 to proximal end 125 of the anchor element 105. However, this design is not necessarily a limiting feature of the invention and one of skill in the art would understand that other leaf configurations are contemplated.

The leaves 130 are shape set to extend past the outer surface of the tube and become the bone-anchoring component of the assembly 100. In a preferred embodiment, the leaves 130 are shape set in a configuration such that one edge or side of the leaf 130 projects radially outward at a greater distance than the opposite edge of the leaf 130. This gives the leaves 130 a radial “wave” or curvilinear shape along the cut edge. In the illustrated embodiment, edge 132 of leaf 130 projects radially outward farther than opposite edge 131. This creates a relatively large opened angle between the edge 132 and the tube wall when compared to the smaller angle between the edge 131 and the tube wall, and allows the anchor element 105 to engage the bone when the edge 132 is rotated into the bone. Referring to the embodiment illustrated in FIGS. 4A and 4B, the anchor element 105 will fully engage and anchor into the bone when the anchor element is rotated clockwise.

This design additionally provides pull-out resistance, and allows the anchor element 105 to engage and anchor into the bone when a pulling force is exerted on the anchor assembly 100. Similar to the anchoring method described above, the pulling motion causes the leading edges 132 of leaves 130 to engage and anchor into the bone.

Once the bone anchor element is formed, the leaves 130 remain in the shape set expanded configuration. As the bone anchor 100 is placed into the core hole drilled in the target bone, the leaves 130 will collapse down to conform to the inside diameter of the core hole. Because the leaves are shape set from a super elastic and shape memory material, they exert a constant outward force against the bone.

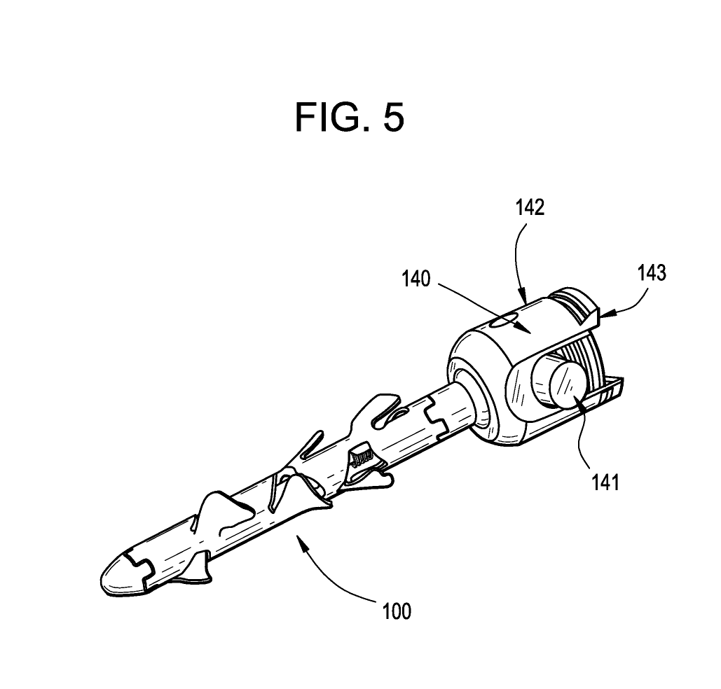

The bone anchor core 110 is a critical component of the assembly 100, tying the anchor element 105 and the anchored medical device. FIG. 5 is a perspective view illustrating the anchor assembly 100 connected to a head 140.

Common spinal fixation techniques involve immobilizing the spine by using orthopedic rods 141, commonly referred to as spine rods, which run generally parallel to the spine. In the illustrated embodiment, spinal fixation would be accomplished by exposing the spine posteriorly or anteriorly (not shown) and fastening the anchor assembly 100 to the pedicles or laminae of the appropriate vertebrae. The anchor assembly 100 is attached to a head assembly 140 that fixes the rod 141 to the anchor assembly 100. The head assembly 140 may be polyaxial (e.g., as described in U.S. Pat. Nos. 5,672,176 (Biedermann) or 6,485,491 (Farris)) or monoaxial (e.g., as described in U.S. Pat. Nos. 5,738,658 (Halm) or 5,725,527 (Biedermann)) types.

Head assemblies, such as axial head 140 are typically comprised of U-shaped receiving elements 142 adapted for receiving the spine rod 141 there through, and join the spine rods 141 to the anchor assembly 100. The aligning influence of the rods 141 force the spine to conform to a more desirable shape. In certain instances, the spine rods 141 may be bent to achieve the desired curvature of the spinal column.

Once the anchor assembly 100 has been implanted, and a spinal rod 141 has been introduced into the receiving element 142 of the head assembly 140, insertion instruments are used to apply a securing screw 143 to the receiver of the anchor assembly 100 to contain the spinal rod 141. A light torque is generally used to first capture the spinal rod 141. Additional torque may be applied to the securing screw 143 if compression and/or distraction are required. Once the surgeon is satisfied with the placement of the spinal rod, the recommended final tightening torque will be applied to the securing screw 143 to secure the spinal rod 141 in place.

These and other objects and advantages of this invention will become obvious to a person of ordinary skill in this art upon reading of the detailed description of this invention including the associated drawings.

Various other modifications, adaptations, and alternative designs are of course possible in light of the above teachings. Therefore, it should be understood at this time that within the scope of the appended claims the invention might be practiced otherwise than as specifically described herein.

What is claimed is:

1) A bone anchor assembly comprising:
An anchor core having a proximal and distal end; and
An elongate tubular anchor element concentrically disposed over and engaged with the anchor core, the anchor element having shape set anchors extending radially outward for engaging with a bone.

2) The bone anchor assembly according to claim 1 wherein said anchor element comprises a metallic tube.

3) The anchor element according to claim 2 wherein said metallic tube comprises nitinol.

4) The anchor element according to claim 3 wherein said tube is in a super elastic state at zero stress.

5) The bone anchor assembly according to claim 1 wherein said anchor element and said shape set anchors are monolithic.

6) The bone anchor assembly according to claim 1 wherein said shape set anchors are normally open radially outward from the outer surface of said anchor element at zero stress.

7) The bone anchor assembly according to claim 1 wherein said shape set anchors collapse radially inward when an external force is applied.

8) The bone anchor assembly according to claim 7 wherein the external force is generated through the insertion of said bone anchor assembly into an opening in the bone.

9) The bone anchor assembly according to claim 8 wherein said shape set anchors conform to and engage with the inner contours of the opening into which said bone anchor assembly is inserted.

10) The bone anchor assembly according to claim 1 wherein said shape set anchors have at least two free sides.

11) The bone anchor assembly according to claim 1 wherein said shape set anchors are arranged in a spiraled configuration about the anchor element such that each shape set anchor is rotationally offset from the distally and proximally adjacent shape set anchor.

12) The bone anchor assembly according to claim 11 wherein said rotational offset of said shape set anchors allow said anchor element to be removed by rotating the anchor element in a known direction.

13) The bone anchor assembly according to claim 1 wherein the said shape set protrusions are shape set to have a curvilinear bias.

14) The bone anchor assembly according to claim 1 wherein said shape set anchors provide a constant outward radial engaging force when subject to an opposing force having a radially compressive component.

15) The bone anchor assembly according to claim 1 wherein said shape set anchors provide engagement force when an axial tensile force is applied toward the proximal end of said anchor element.

16) The bone anchor assembly according to claim 1 wherein said anchor element possesses at least one detent cut out of each of said proximal and distal ends.

17) The bone anchor assembly according to claim 1 wherein said anchor core comprises a biocompatible material.

18) The anchor core according to claim 17 wherein said biocompatible material comprises titanium.

19) The anchor core according to claim 17 wherein said biocompatible material comprises stainless steel.

20) The anchor core according to claim 17 wherein said biocompatible material comprises plastic.

21) The anchor core according to claim 17 wherein said biocompatible material comprises ceramic.

22) The anchor core according to claim 17 wherein said biocompatible material comprises a composite.

23) The bone anchor assembly according to claim 1 wherein said anchor core supports the anchor element.

24) The bone anchor assembly according to claim 1 wherein the proximal end of said anchor core is adapted to be secured to a medical device.

25) The bone anchor assembly according to claim 1 wherein the proximal end of said anchor core is spherically shaped.

26) The bone anchor assembly according to claim 1 wherein the proximal end of said anchor core is adapted to be secured to a suture.

27) The bone anchor assembly according to claim 1 wherein the anchor core is comprised of a proximal core and a distal core, said proximal and distal cores being adapted to engage one another at a common point.

28) The bone anchor assembly according to claim 16 wherein said proximal end of the core includes a cog adapted to engage with the detent located on the proximal end of the anchor element.

29) The bone anchor assembly according to claim 28 wherein said cog transmits rotational energy to said anchor element.

30) The bone anchor assembly according to claim 16 wherein said distal end of the core includes a cog adapted to engage with the detent located on the distal end of the anchor element.

31) The bone anchor assembly according to claim 30 wherein said cog transmits rotational energy to said anchor element.

32) The bone anchor assembly according to claim 1 wherein said distal end of said anchor core possesses a conical taper.

33) The bone anchor assembly according to claim 1 wherein said distal end of said anchor core is adapted to be inserted into an opening in the bone.

34) The bone anchor assembly according to claim 1 wherein said anchor element is adapted to secure to an opening in the bone in the absence of threads tapped into the bone opening.

35) An anchor assembly for anchoring in a substantially rigid material comprising:
An anchor core; and
an anchor element concentrically disposed over and engaged with the anchor core, the anchor element having shape set anchors extending radially outward for engaging with an opening in said substantially rigid material.

36) A method of securing a medical device to a bone comprising the steps of:
making a hole in a bone, said hole being sized to operably accept a bone anchor assembly having a plurality of anchor elements disposed about the exterior surface of said bone anchor assembly;
linearly inserting said bone anchor assembly into the opening in the bone without tapping threads into the wall of said hole until said anchor elements are operably engaged with the bone; and
securing the medical device to the distal portion of said bone anchor assembly.

37) The method according to claim 36 wherein the depth of the linear insertion of said bone anchor assembly is adjusted by rotational retraction.

38) A method of using the anchor assembly comprising the steps of:
of making a hole in a substantially rigid material, said hole being sized to operably accept an anchor assembly having a plurality of anchor elements disposed about the exterior surface of said anchor assembly;
linearly inserting said anchor assembly into the opening of the hole, without axial rotation, until said anchor elements are operably engaged with the substantially rigid material.
